(19) United States
(12) Patent Application Publication
(10) Pub. No.: US 2024/0245541 A1
(43) Pub. Date: Jul. 25, 2024

(54) APPARATUS FOR ASSISTING ACTIVE MOTION OF FINGER

(71) Applicant: SOONCHUNHYANG UNIVERSITY INDUSTRY ACADEMY COOPERATION FOUNDATION, Asan-si (KR)

(72) Inventors: Dae Geun KIM, Gumi-si (KR); Hyo Jun PARK, Busan (KR)

(73) Assignee: SOONCHUNHYANG UNIVERSITY INDUSTRY ACADEMY COOPERATION FOUNDATION, Asan-si (KR)

(21) Appl. No.: 18/420,939

(22) Filed: Jan. 24, 2024

(30) Foreign Application Priority Data
Jan. 25, 2023 (KR) 10-2023-0009739

(51) Int. Cl. A61F 5/01

(52) U.S. Cl. CPC A61F 5/0118

(57) ABSTRACT

Disclosed is an apparatus for assisting an active motion of a finger including a standard stabilizer, a hyperextension-specific stabilizer having a shape being bent in a first direction, in which the finger is extended, an active motion-specific elastic part that generates an elastic force in the first direction or a second direction being an opposite direction to the first direction, and a mounting part, to which one of the standard stabilizer, the hyperextension-specific stabilizer, and the active motion-specific elastic part is selectively coupled to be separable, and on which the finger is worn.

### CROSS-REFERENCE TO RELATED APPLICATIONS

A claim for priority under 35 U.S.C. § 119 is made to Korean Patent Application No. 10-2023-0009739 filed on Jan. 25, 2023 in the Korean Intellectual Property Office, the entire contents of which are hereby incorporated by reference.

### BACKGROUND

Embodiments of the present disclosure described herein relate to an apparatus for assisting an active motion of a finger.

Hands are the most important yet most vulnerable parts of a human body. The hands may perform various activities with fine senses and delicate manipulations, but injuries frequently occur as they are used a lot in the human body.

According to a 2009 U.S. statistical survey, among hand injuries, finger fractures are the most common.

Finger fractures may lead to complications, such as a finger joint stiffness, a finger malunion, a finger nonunion, and an infection, and among them, the finger joint stiffness is the most common. This kind of finger joint stiffness is very difficult to treat.

To treat a fracture of the finger, when open reduction and internal fixation or ligament repair is performed on the fractured part of the finger, an extension delay, by which the proximal interphalangeal joint of the finger is hardened to a state of about 30° flexion, may occur.

Accordingly, when open reduction and internal fixation or ligament suturing is performed on the fractured part of the finger, an active motion of the finger is allowed within a range that may prevent displacement of the fractured part of the finger after the finger is fixed for an appropriate period of time. Through this, it is possible to prevent a joint stiffness of the finger.

Meanwhile, a conventional finger splint device may fix the finger for an appropriate period of time, but an active motion of the finger cannot be performed.

### SUMMARY

An aspect of the present disclosure provides an apparatus for assisting an active motion of a finger, by which a finger may be fixed and an active motion of the finger may be performed at the same time.

The problems that the present disclosure seeks to solve are not limited to the problems mentioned above, and other problems not mentioned may be clearly understood by an ordinary person in the art from the description below.

According to an aspect of the present disclosure, an apparatus for assisting an active motion of a finger includes a standard stabilizer, a hyperextension-specific stabilizer having a shape being bent in a first direction, in which the finger is extended, an active motion-specific elastic part that generates an elastic force in the first direction or a second direction being an opposite direction to the first direction, and a mounting part, to which one of the standard stabilizer, the hyperextension-specific stabilizer, and the active motion-specific elastic part is selectively coupled to be separable, and on which the finger is worn.

Furthermore, the mounting part may include a first mounting body mounted on a distal part of the finger, and a second mounting body connected to the first mounting body to be rotatable, and mounted on a central part of the finger, and the apparatus may include a first rotation limiting part provided between the first mounting body and the second mounting body, and that limits a first directional rotation range of the first mounting body.

Furthermore, the first rotation limiting part may include a first inclined part formed on one side of a rear side of the first mounting body to be inclined, and a first auxiliary inclined part formed on one side of a front side of the second mounting body to be inclined.

Furthermore, the mounting part may include a third mounting body connected to the second mounting body to be rotatable, and mounted on a proximal part of the finger, and a second rotation limiting part provided between the second mounting body and the third mounting body, and that limits the first directional rotation range of the second mounting body.

Furthermore, the second rotation limiting part may include a second inclined part formed on one side of a rear side of the second mounting body to be inclined, and a second auxiliary inclined part formed on one side of a front side of the third mounting body to be inclined.

Furthermore, the standard stabilizer may have a plurality of standard fixing bosses, a plurality of hyperextension-specific fixing bosses may be formed in the hyperextension-specific stabilizer, a plurality of active motion-specific fixing bosses may be formed in the active motion-specific elastic part, and a plurality of fixing recesses, to which any one group of the plurality of standard fixing bosses, the plurality of hyperextension-specific fixing bosses, and the plurality of active motion-specific fixing bosses is coupled to be separable, may be formed in the mounting part.

Furthermore, the mounting part may include a first link connecting the first mounting body and the second mounting body such that the first mounting body and the second mounting body are rotatable, and a second link connecting the second mounting body and the third mounting body such that the second mounting body and the third mounting body are rotatable.

Furthermore, a first track may be formed in the first mounting body, a second track and a second auxiliary track may be formed on a front side and a rear side of the second mounting body, respectively, a third track may be formed in the third mounting body, opposite sides of the first link may be coupled to be movable along the first track and the second track and rotatable, respectively, and opposite sides of the second link may be coupled to be movable along the second auxiliary track and the third track and rotatable, respectively.

Other details of the present disclosure are included in the detailed description and the drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a state, in which a standard stabilizer is coupled to a mounting part of an apparatus for assisting an active motion of a finger according to an embodiment of the present disclosure;

FIG. 2 is a perspective view illustrating a state, in which a second link is disassembled from a mounting part of an apparatus for assisting an active motion of a finger according to an embodiment of the present disclosure;

FIG. 3 is a perspective view illustrating a state, in which a standard stabilizer is separated from a mounting part of an apparatus for assisting an active motion of a finger according to an embodiment of the present disclosure;

FIG. 4 is a perspective view illustrating a state, in which a hyperextension-specific stabilizer is coupled to a mounting part of an apparatus for assisting an active motion of a finger according to an embodiment of the present disclosure;

FIG. 5 is a perspective view illustrating a state, in which a hyperextension-specific stabilizer is separated from a mounting part of an apparatus for assisting an active motion of a finger according to an embodiment of the present disclosure;

FIG. 6 is a perspective view illustrating a state, in which a bending-specific elastic part is coupled to a mounting part of an apparatus for assisting an active motion of a finger according to an embodiment of the present disclosure;

FIG. 7 is a perspective view illustrating a state, in which a bending-specific elastic part is separated from a mounting part of an apparatus for assisting an active motion of a finger according to an embodiment of the present disclosure;

FIG. 8 is a perspective view illustrating a state, in which a standard stabilizer is coupled to a first mounting body and a second mounting body of a mounting part of an apparatus for assisting an active motion of a finger according to an embodiment of the present disclosure;

FIG. 9 is a perspective view illustrating a state, in which a standard stabilizer is separated from the a first mounting body and a second mounting body of a mounting part of an apparatus for assisting an active motion of a finger according to an embodiment of the present disclosure; and

FIG. 10 is a perspective view illustrating a hyperextension-specific stabilizer separated from a first mounting body and a second mounting body of a mounting part of an apparatus for assisting an active motion of a finger according to an embodiment of the present disclosure.

### DETAILED DESCRIPTION

The advantages and features of the present disclosure, and a method for achieving them will become clear with reference to the embodiments that will be described in detail together with the accompanying drawings. However, the present disclosure is not limited by the embodiments disclosed hereinafter but may be implemented in various different forms, and the embodiments are provided simply to make the disclosure of the present disclosure complete and inform an ordinary person in the art of the scope of the present disclosure, and the disclosure is only defined by the scope of the claims.

The terms used in the specification is for describing the embodiments, and is not intended to limit the present disclosure. A singular expression includes a plural expression unless an exemption is particularly described in the specification. The expression “comprises” and/or “comprising” used in the specification does not exclude presence or addition of one or more other components, in addition to the mentioned components. Throughout the specification, the same reference numerals denote the same components, and the term “and/or” includes one or more combinations of the mentioned components. Although “first”, “second”, or the like is used to describe various components, it is apparent that the components are not limited by the terms. The terms are used simply to distinguish one component from another component. Accordingly, it is apparent that a first component mentioned hereinafter may be a second component in the technical spirit of the present disclosure.

Unless otherwise defined, all terms (including technical and scientific terms) used herein have the same meaning as commonly understood by those skilled in the art, to which the present disclosure pertains. Furthermore, the terms defined in commonly used dictionaries should not be interpreted in an idealized or overly formal sense unless expressly so defined herein.

Hereinafter, embodiments of the present disclosure will be described in detail with reference to the attached drawings. In indicating directions of the present disclosure, a first direction may be a clockwise direction, and a second direction may be a counterclockwise direction. Furthermore, a forward/rearward direction, a forward direction, and a rearward direction may be a forward/rearward direction, a forward direction, and a rearward direction in the drawings.

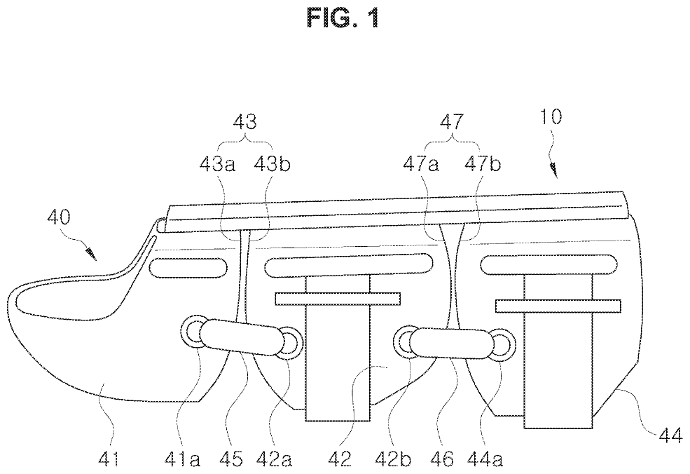

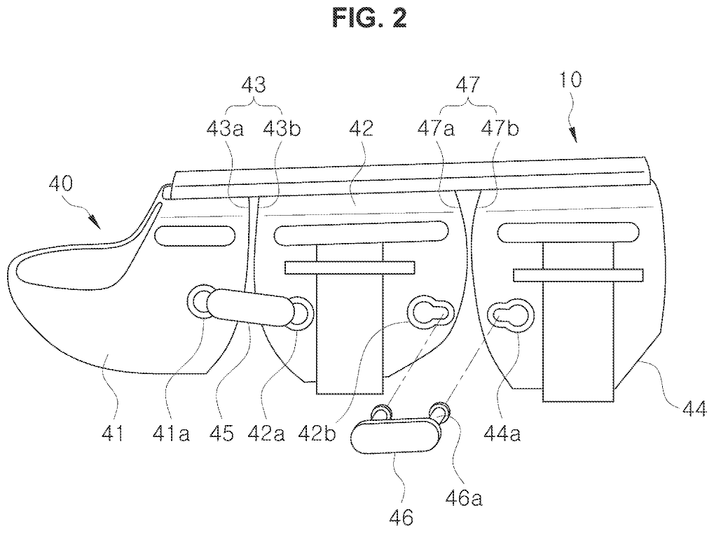

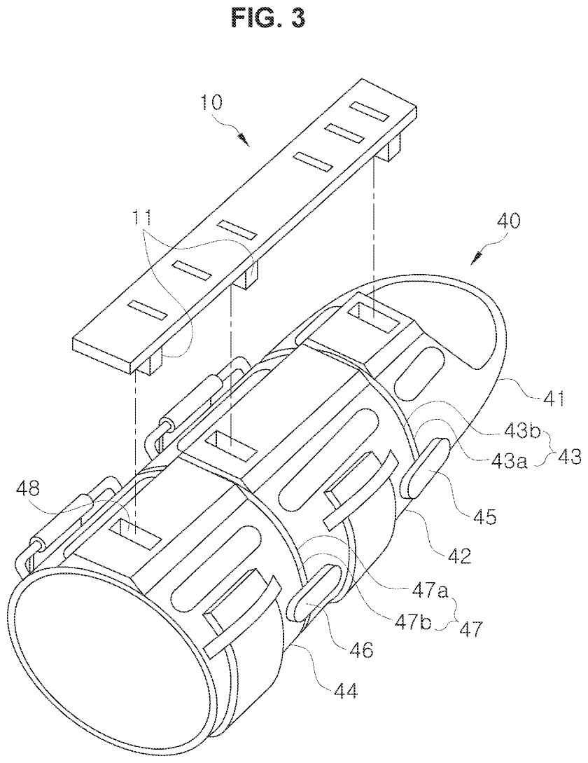

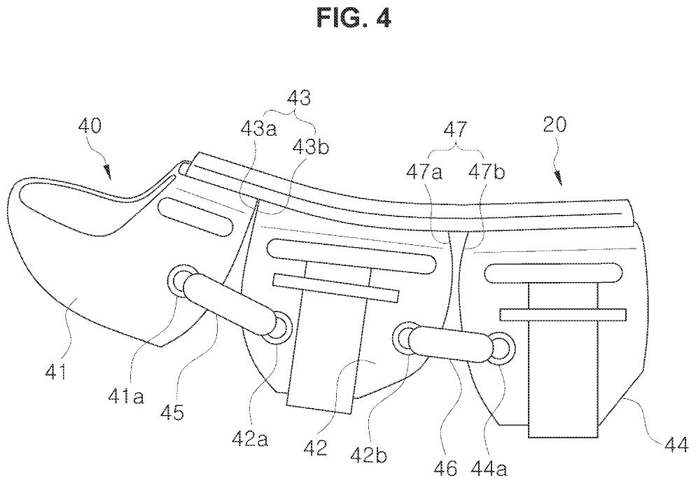

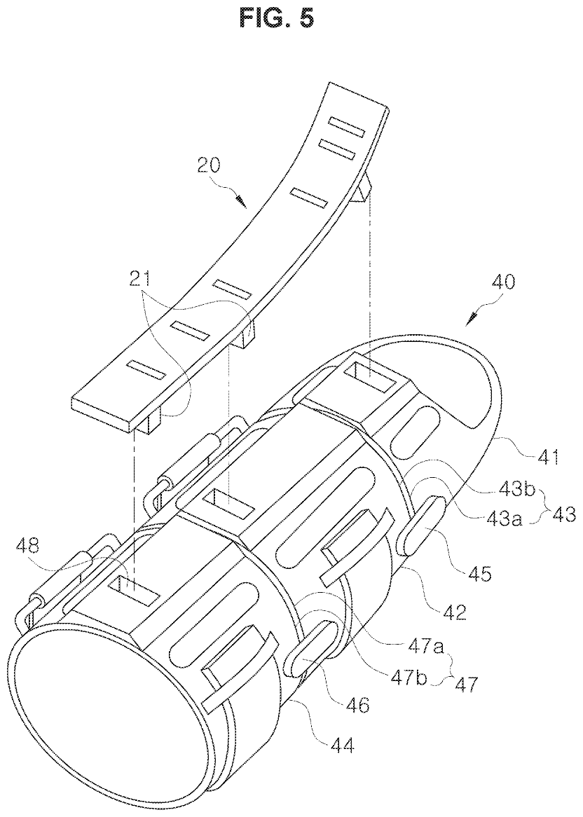

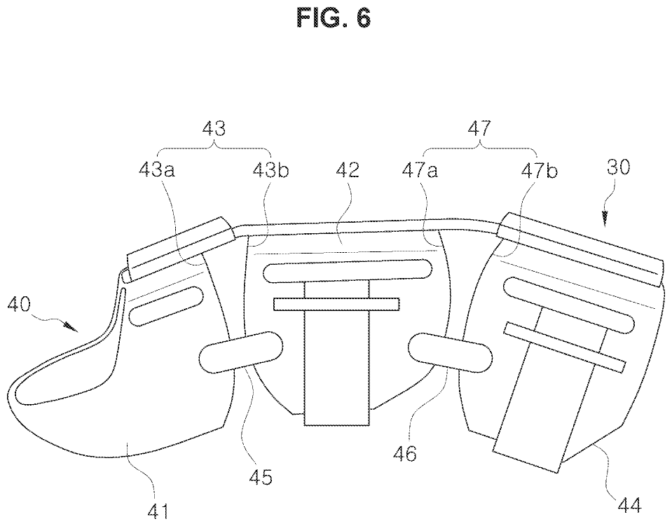

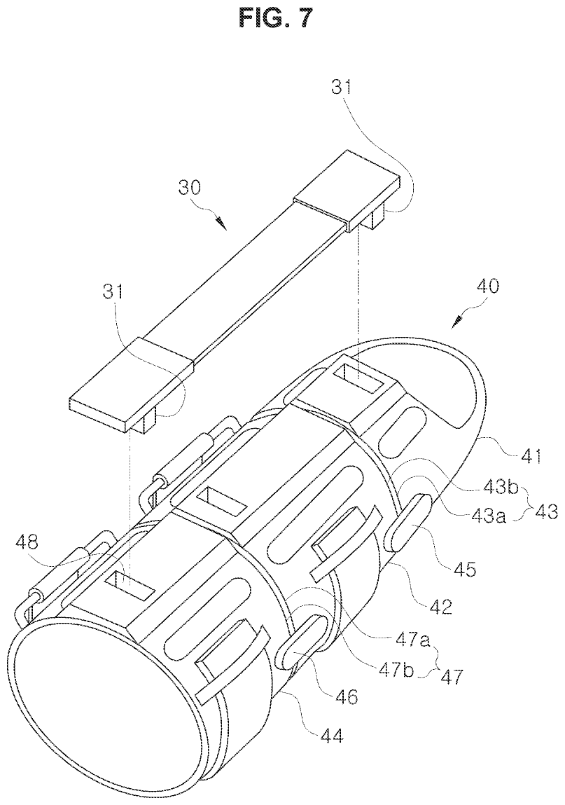

FIG. 1 is a perspective view illustrating a state, in which a standard stabilizer 10 is coupled to a mounting part 40 of an apparatus for assisting an active motion of a finger according to an embodiment of the present disclosure, FIG. 2 is a perspective view illustrating a state, in which a second link 46 is disassembled from the mounting part 40 of the apparatus for assisting an active motion of a finger according to an embodiment of the present disclosure, FIG. 3 is a perspective view illustrating a state, in which the standard stabilizer 10 is separated from the mounting part 40 of the apparatus for assisting an active motion of a finger according to an embodiment of the present disclosure, FIG. 4 is a perspective view illustrating a state, in which a hyperextension-specific stabilizer 20 is coupled to the mounting part 40 of the apparatus for assisting an active motion of a finger according to an embodiment of the present disclosure, FIG. 5 is a perspective view illustrating a state, in which the hyperextension-specific stabilizer 20 is separated from the mounting part 40 of the apparatus for assisting an active motion of a finger according to an embodiment of the present disclosure, FIG. 6 is a perspective view illustrating a state, in which a bending-specific elastic part is coupled to the mounting part 40 of the apparatus for assisting an active motion of a finger according to an embodiment of the present disclosure, and FIG. 7 is a perspective view illustrating a state, in which the bending-specific elastic part is separated from the mounting part 40 of the apparatus for assisting an active motion of a finger according to an embodiment of the present disclosure.

As illustrated in FIGS. 1 to 6, the apparatus for assisting an active motion of a finger according to an embodiment of the present disclosure includes the standard stabilizer 10, the hyperextension-specific stabilizer 20, and an active motion-specific elastic part 30, and the mounting part 40.

Referring to FIGS. 1 and 3, the standard stabilizer 10 is coupled to the mounting part 40, on which a finger is worn, to be separable, and may serve to fix the mounting part 40 in a horizontal state. Then, the finger worn on the mounting part 40 may be maintained in a horizontal state. In this way, when the standard stabilizer 10 is coupled to the mounting part 40, on which the finger is worn, a fastening force may be maintained between a plurality of fixing pins (not illustrated) that are fixed to a joint of the finger worn on this mounting part 40 and the joint of the finger.

As an example, the standard stabilizer 10 may have a bar shape.

As another example, a plurality of standard fixing bosses 11 may be formed in the standard stabilizer 10. The plurality of standard fixing bosses 11 may be coupled to the plurality of fixing recesses 48 of the mounting part 40, which will be described later, to be separable. Furthermore, the plurality of standard fixing bosses 11 may have a plate shape. Furthermore, the standard fixing bosses 11 may extend vertically from the standard stabilizer 10.

Meanwhile, the standard stabilizer 10 may be coupled along a first mounting body 41, a second mounting body 42, and a third mounting body 44 of the mounting part 40, which will be described later, it may serve to fix the first mounting body 41, the second mounting body 42, and the third mounting body 44 in a horizontal state.

Referring to FIGS. 4 and 5, the hyperextension-specific stabilizer 20 may be coupled to the mounting part 40, on which the finger is worn, to be separable and may serve to fix the mounting part 40 in a bent state in a first direction. Then, the finger worn on the mounting part 40 may be fixed in a hyperextension state. In this way, when the hyperextension-specific stabilizer 20 is coupled to the mounting part 40, on which the finger is worn, it is possible to cope with a mallet finger symptom that requires a hyperextension of the finger worn on this mounting part 40. The hyperextension-specific stabilizer 20 may limit a rotation range of the mounting part 40, on which the finger is worn, to within 15° to 20°. Through this, the hyperextension-specific stabilizer 20 may improve an effect of coping with the mallet finger symptom of the finger worn on the mounting part 40.

As an example, the hyperextension-specific stabilizer 20 may have a shape that is bent in the first direction, in which the finger is extended. In detail, the hyperextension-specific stabilizer 20 may have a shape, in which a front side of the hyperextension-specific stabilizer 20 is more bent in the first direction than a rear side of the hyperextension-specific stabilizer 20.

As another example, a plurality of hyperextension-specific fixing bosses 21 may be formed in the hyperextension-specific stabilizer 20. The plurality of hyperextension-specific fixing bosses 21 may be coupled to the plurality of fixing recesses 48 of the mounting part 40, which will be described later, to be separable. Furthermore, the hyperextension-specific fixing boss 21 may have a plate shape. Furthermore, the hyperextension-specific fixing boss 21 may extend vertically from the hyperextension-specific stabilizer 20.

Meanwhile, the hyperextension-specific stabilizer 20 may be coupled along the first mounting body 41, the second mounting body 42, and the third mounting body 44 of the mounting part 40, which will be described later, and may serve to fix the first mounting body 41, the second mounting body 42, and the third mounting body 44 in a bent state in the first direction.

Referring to FIGS. 6 and 7, the active motion-specific elastic part 30 may be coupled to the mounting part 40, on which the finger is worn, to be separable to deliver an elastic force in a second direction that is an opposite direction to the first direction and in which the finger is bent, to the mounting part 40. Then, the finger worn on the mounting part 40 may receive an assistant force that assists an active motion by the elastic force of the active motion-specific elastic part 30. In this way, when the active motion-specific elastic part 30 is coupled to the mounting part 40, on which the finger is worn, to be separable, a dynamic exercise function for preventing a joint stiffness and a contracture of the finger worn on the mounting part 40 may be implemented. This active motion-specific elastic part 30 may also serve to limit a rotation range of the finger worn on the mounting part 40 to within 130°.

As an example, a plurality of active motion-specific fixing bosses 31 may be formed in the active motion-specific elastic part 30. The plurality of active motion-specific fixing bosses 31 may be coupled to the plurality of fixing recesses 48 of the mounting part 40, which will be described later, to be separable. Furthermore, the plurality of active motion-specific fixing bosses 31 may have a plate shape. Furthermore, the plurality of active motion-specific fixing bosses 31 may extend vertically from the active motion-specific elastic part 30.

Meanwhile, the active motion-specific elastic part 30 may be coupled along the first mounting body 41, the second mounting body 42, and the third mounting body 44 of the mounting part 40, which will be described later, and may provide an elastic force to the first mounting body 41, the second mounting body 42, and the third mounting body 44 of the mounting part 40 in the first or second direction.

The mounting part 40 may be selectively coupled to one of the standard stabilizer 10, the hyperextension-specific stabilizer 20, and the active motion-specific elastic part 30 to be separable, and may be mounted on the finger.

The mounting part 40 may include the first mounting body 41, the second mounting body 42, a first rotation limiting part 43, the third mounting body 44, and a second rotation limiting part 47.

The first mounting body 41 may be mounted on a distal part of the finger. A rear side of the first mounting body 41 may be connected to a front side of the second mounting body 42, which will be described later, to be rotatable.

The second mounting body 42 may be connected to the first mounting body 41 to be rotatable, and may be mounted on the central part of the finger. A front side of the second mounting body 42 may be connected to the rear side of the first mounting body 41 to be rotatable, and a rear side of the second mounting body 42 may be connected to a front side of the third mounting body 44 to be rotatable.

Meanwhile, the first mounting body 41 and the second mounting body 42 may be connected to each other to be rotatable through a first link 45.

The first link 45 may connect the first mounting body 41 and the second mounting body 42 such that the first mounting body 41 and the second mounting body 42 are rotatable. As an example, a first track 41a may be formed at a rear side of the first mounting body 41, a second track 42a may be formed at a front side of the second mounting body 42, and opposite sides of the first link 45 may be coupled to be movable along the first track 41a and the second track 42a and rotatable. Accordingly, the first mounting body 41 and the second mounting body 42 may be moved in a direction, in which approach each other or becomes farther away from each other, through a coupling relationship between the first link 45, the first track 41a, and the second track 42a. Furthermore, the first mounting body 41 and the second mounting body 42 may be rotated through the coupling relationship between the first link 45, the first track 41a, and the second track 42a.

As an example, a pair of multi-stage first insertion shafts (not illustrated) may be provided on both sides of the first link 45, and the pair of first insertion shafts may be coupled along the first track 41a and the second track 42a, respectively, to be movable.

The first rotation limiting part 43 may be provided on the first mounting body 41 and the second mounting body 42, and may serve to limit the first directional rotation range of the first mounting body 41. The first rotation limiting part 43 may include a first inclined part 43a and a first auxiliary inclined part 43b.

The first inclined part 43a may be formed on one side of a rear side of the first mounting body 41, which faces the second mounting body 42 to be inclined. Here, one side of a rear side of the first mounting body 41, which faces the second mounting body 42, may be a rear upper side of the first mounting body 41, which faces the second mounting body 42.

The first auxiliary inclined part 43b may be formed on one side of a front side of the second mounting body 42, which faces the first mounting body 41, to be inclined. Here, one side of a front side of the second mounting body 42, which faces the first mounting body 41, may be a front upper side of the second mounting body 42, which faces the first mounting body 41.

A scheme of limiting a first directional rotation range of the first mounting body 41 by the first inclined part 43a and the first auxiliary inclined part 43b will be described as follows. In a state, in which the first mounting part 40 and the second mounting part 40 are disposed horizontally, as the first inclined part 43a provided in the first mounting body 41 contacts the first auxiliary inclined part 43b provided in the second mounting body 42 when the first mounting body 41 approaches the second mounting body 42 while being rotated in the first direction, a rotation range of the first mounting body 41 in the first direction may be limited.

The third mounting body 44 may be connected to the second mounting body 42 to be rotatable, and may be mounted on a proximal part of the finger. A front side of the third mounting body 44 may be connected to a rear side of the second mounting body 42 to be rotatable.

Meanwhile, the second mounting body 42 and the third mounting body 44 may be connected to each other to be rotatable through the second link 46.

The second link 46 may connect the second mounting body 42 and the third mounting body 44 such that the second mounting body 42 and the third mounting body 44 are rotatable. As an example, a second auxiliary track 42b may be formed on a rear side of the second mounting body 42, a third track 44a may be formed on a front side of the third mounting body 44, and opposite sides of the second link 46 may be coupled to be movable along the second auxiliary track 42b and the third track 44a. Accordingly, the second mounting body 42 and the third mounting body 44 may be moved in a direction, in which they approach each other or become farther away from each other, through a coupling relationship between the second link 46, the second auxiliary track 42b, and the third track 44a. Furthermore, the second mounting body 42 and the third mounting body 44 may be rotated through a coupling relationship between the second link 46, the second auxiliary track 42b, and the third track 44a.

As an example, a pair of multi-stage second insertion shafts 46a may be provided on opposite sides of the second link 46, and the pair of second insertion shafts 46a may be coupled to be movable along the second auxiliary track 42b and the third track 44a, respectively.

The second rotation limiting part 47 may be provided in the second mounting body 42 and the third mounting body 44, and may serve to limit the first directional rotation range of the second mounting body 42. The second rotation limiting part 47 may include a second inclined part 47a and a second auxiliary inclined part 47b.

The second inclined part 47a may be formed on one side of a rear side of the second mounting body 42, which faces the third mounting body 44, to be inclined. Here, the one side of the rear side of the second mounting body 42, which faces the third mounting body 44, may be a rear upper side of the second mounting body 42, which faces the third mounting body 44.

The second auxiliary inclined part 47b may be formed on one side of a front side of the third mounting body 44, which faces the second mounting body 42, to be inclined. Here, one side of a front side of the third mounting body 44, which faces the second mounting body 42, may be a front upper side of the third mounting body 44, which faces the second mounting body 42.

Hereinafter, an example of using the apparatus for assisting an active motion of a finger according to an embodiment of the present disclosure will be described. The following process may be performed by a user or a robot arm.

First, when a finger that has undergone open reduction, internal fixation, or ligament suturing is coupled to the mounting part 40, the standard stabilizer 10 is coupled to the mounting part 40, on which the finger is worn. In this way, the standard stabilizer 10 is coupled to the mounting part 40, on which the finger is worn, a fastening force may be maintained between the plurality of fixing pins (not illustrated) fixed to the joint of the finger worn on the mounting part 40 and the joint of the finger.

Next, the standard stabilizer 10 is removed from the mounting part 40, on which the finger is worn, and the active motion-specific elastic part 30 is attached to the mounting part 40, on which the finger is worn. In this way, because the active motion-specific elastic part 30 is coupled to the mounting part 40, on which the finger is worn, to be separable, a dynamic exercise function for prevent a joint stiffness and a contracture of the finger worn on the mounting part 40 may be implemented.

Hereinafter, another use example of the apparatus for assisting an active motion of a finger according to an embodiment of the present disclosure will be described. The following process may be performed by the user or the robot arm.

When a finger with a mallet finger symptom is coupled to the mounting part 40, the hyperextension-specific stabilizer 20 is attached to the mounting part 40, on which the finger is worn. In this way, when the hyperextension-specific stabilizer 20 is coupled to the mounting part 40, on which the finger is worn, it is possible to cope with the mallet finger symptom that requires a hyperextension of the finger worn on this mounting part 40.

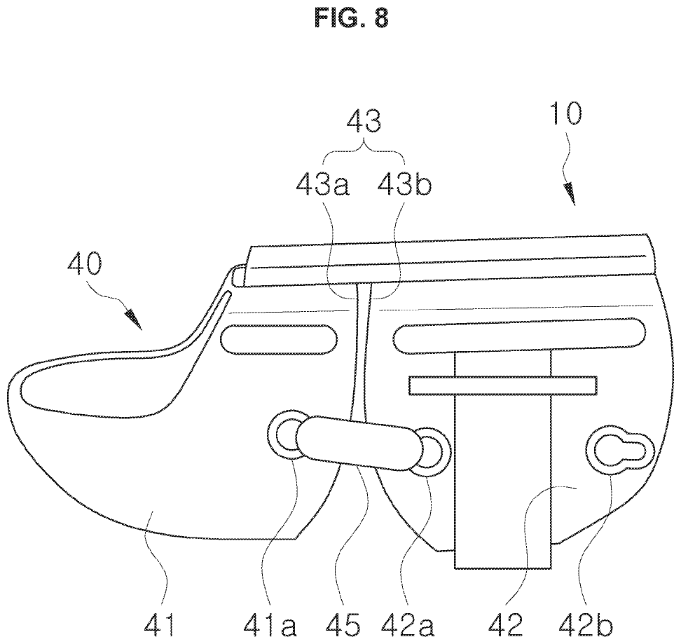

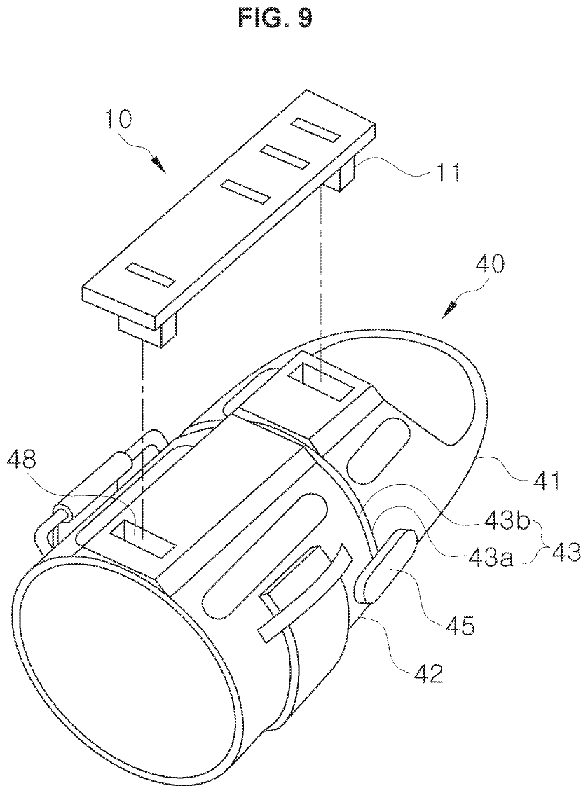

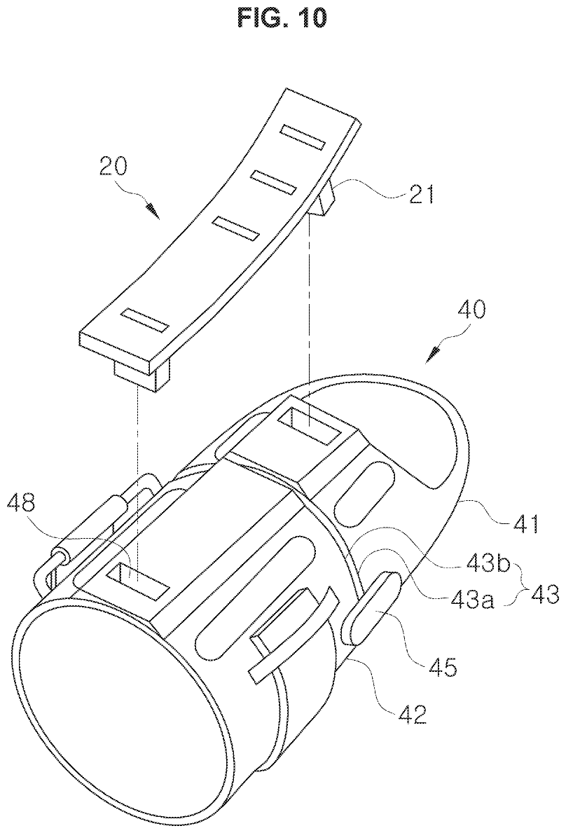

FIG. 8 is a perspective view illustrating a state, in which the standard stabilizer 10 is coupled to the first mounting body 41 and the second mounting body 42 of the mounting part 40 of the apparatus for assisting an active motion of a finger according to an embodiment of the present disclosure, FIG. 9 is a perspective view illustrating a state, in which the standard stabilizer 10 is separated from the first mounting body 41 and the second mounting body 42 of the mounting part 40 of the apparatus for assisting an active motion of a finger according to an embodiment of the present disclosure, and FIG. 10 is a perspective view illustrating the hyperextension-specific stabilizer 20 separated from the first mounting body 41 and the second mounting body 42 of the mounting part 40 of the apparatus for assisting an active motion of a finger according to an embodiment of the present disclosure.

As illustrated in FIG. 8, the standard stabilizer 10 may be coupled only along the first mounting body 41 and the second mounting body 42 of the mounting part 40, and the third mounting body 44 may be excluded. Alternatively, the standard stabilizer 10 may be coupled only along the second mounting body 42 and the third mounting body 44 of the mounting part 40, and the first mounting body 41 may be excluded.

As illustrated in FIGS. 9 and 10, the hyperextension-specific stabilizer 20 may be coupled only along the first mounting body 41 and the second mounting body 42 of the mounting part 40, and the third mounting body 44 may be excluded. Alternatively, the hyperextension-specific stabilizer 20 may be coupled only along the second mounting body 42 and the third mounting body 44 of the mounting part 40, and the first mounting body 41 may be excluded.

Although not illustrated in the drawing, the active motion-specific elastic part 30 may be coupled only along the first mounting body 41 and the second mounting body 42 of the mounting part 40, and the third mounting body 44 may be excluded. Alternatively, the active motion-specific elastic part 30 may be coupled only along the second mounting body 42 and the third mounting body 44 of the mounting part 40, and the first mounting body 41 may be excluded.

According to the present disclosure, the apparatus for assisting an active motion of a finger according to an embodiment of the present disclosure may fix the finger and perform an active motion of the finger at the same time.

The effects of the present disclosure are not limited to the effects mentioned above, and other effects not mentioned may be clearly understood by an ordinary person in the art from the description below.

Until now, the embodiments of the present disclosure have been described with reference to the accompanying drawings, but it may be understood that an ordinary person in the art, to which the present disclosure pertains, may carry out the present disclosure in another specific form while not changing the technical spirit or the essential features. Therefore, it should be understood that the embodiments described above are all illustrative, and not restrictive.

What is claimed is:

1. An apparatus for assisting an active motion of a finger, the apparatus comprising:
a standard stabilizer;
a hyperextension-specific stabilizer having a shape being bent in a first direction, in which the finger is extended;
an active motion-specific elastic part configured to generate an elastic force in the first direction or a second direction being an opposite direction to the first direction; and
a mounting part, to which one of the standard stabilizer, the hyperextension-specific stabilizer, and the active motion-specific elastic part is selectively coupled to be separable, and on which the finger is worn,
wherein the mounting part includes:
a first mounting body mounted on a distal part of the finger; and
a second mounting body connected to the first mounting body to be rotatable, and mounted on a central part of the finger, and
wherein the apparatus includes a first rotation limiting part provided between the first mounting body and the second mounting body, and configured to limit a first directional rotation range of the first mounting body.

2. The apparatus of claim 1, wherein the first rotation limiting part includes:
a first inclined part formed on one side of a rear side of the first mounting body to be inclined; and
a first auxiliary inclined part formed on one side of a front side of the second mounting body to be inclined.

3. The apparatus of claim 1, wherein the mounting part includes:
a third mounting body connected to the second mounting body to be rotatable, and mounted on a proximal part of the finger; and
a second rotation limiting part provided between the second mounting body and the third mounting body, and configured to limit the first directional rotation range of the second mounting body.

4. The apparatus of claim 3, wherein the second rotation limiting part includes:
a second inclined part formed on one side of a rear side of the second mounting body to be inclined; and
a second auxiliary inclined part formed on one side of a front side of the third mounting body to be inclined.

5. The apparatus of claim 1, wherein the standard stabilizer has a plurality of standard fixing bosses,
wherein a plurality of hyperextension-specific fixing bosses are formed in the hyperextension-specific stabilizer,
wherein a plurality of active motion-specific fixing bosses are formed in the active motion-specific elastic part, and
wherein a plurality of fixing recesses, to which any one group of the plurality of standard fixing bosses, the plurality of hyperextension-specific fixing bosses, and the plurality of active motion-specific fixing bosses is coupled to be separable, are formed in the mounting part.

6. The apparatus of claim 3, wherein the mounting part includes:
a first link connecting the first mounting body and the second mounting body such that the first mounting body and the second mounting body are rotatable; and
a second link connecting the second mounting body and the third mounting body such that the second mounting body and the third mounting body are rotatable.

7. The apparatus of claim 6, wherein a first track is formed in the first mounting body,
wherein a second track and a second auxiliary track are formed on a front side and a rear side of the second mounting body, respectively,
wherein a third track is formed in the third mounting body,
wherein opposite sides of the first link are coupled to be movable along the first track and the second track and rotatable, respectively, and
wherein opposite sides of the second link are coupled to be movable along the second auxiliary track and the third track and rotatable, respectively.
